(19) United States
(12) Patent Application Publication
(10) Pub. No.: US 2004/0091874 A1
(43) Pub. Date: May 13, 2004

(54) Sensor chip for nucleic acid selection

(75) Inventors: Kazuhiko Yamasaki, Ibaraki (JP); Dongyun Hao, Ibaraki (JP)

Correspondence Address: FOLEY AND LARDNER, SUITE 500, 3000 K STREET NW, WASHINGTON, DC, 20007, US

(73) Assignee: National Institute of Advanced Industrial Science and Technology

(21) Appl. No.: 10/301,875

(22) Filed: Nov. 22, 2002

(30) Foreign Application Priority Data
May 23, 2002 (JP) 2002-149330

(51) Int. Cl. C12Q 1/68; C12M 1/34

(52) U.S. Cl. 435/6; 435/287.2

(57) ABSTRACT

A sensor chip for surface plasmon resonance measurement used for selecting nucleic acids that bind to polypeptides, which has NTA group on its surface for immobilizing polypeptides.

### FIELD OF THE INVENTION

The present invention relates to a sensor chip for surface plasmon resonance measurement for selecting nucleic acids that binds to polypeptides, and a method for selecting nucleic acids using the same.

### BACKGROUND OF THE INVENTION

Molecules such as a DNA-binding protein which specifically binds to a nucleic acid recognize a nucleic acid sequence, and express their functions. Determining a nucleic acid sequence that is recognized by a DNA-binding protein is extremely important to know the functions of the protein. So far determining a sequence has been performed in such a manner comprising (a) selecting nucleic acids which can bind to proteins or the like from various nucleic acids molecules having randomized sequences; (b) amplifying the selected nucleic acids by the polymerase chain reaction (PCR); (c) further selection and amplification, i.e., repeating (a) and (b), to increase the percentage of binding nucleic acid molecules; (d) finally determining a sequence of the selected nucleic acid.

In such sequencing, examples of the above methods for selecting a nucleic acid that binds to the protein include: (1) a method which comprises contacting a solution of the various nucleic acids molecules with a column or beads having protein immobilized thereto, washing off unbound nucleic acids, and then eluting only bound nucleic acids, (2) a method which comprises mixing protein with a solution of the various nucleic acids molecules, contacting the solution with a nitrocellulose membrane having high affinity for protein, washing off unbound nucleic acids, and then eluting only bound nucleic acids, and (3) a method which comprises excising a band corresponding to a protein-nucleic acid complex, by polyacrylamide gel electrophoresis.

However, with these conventional methods, it has not been possible to obtain, in real-time during experiments, information concerning immobilization of proteins and the binding states of nucleic acids. Accordingly, it has not been easy to determine whether or not a sufficient amount of protein is immobilized, or whether or not protein and nucleic acid are really bound to each other via the nucleic acid recognition site of the protein. Moreover, because of such difficulties in determination, it has not been easy to adjust experimental conditions appropriate for immobilizing the protein or binding the nucleic acid. Hence, there has been a risk of continuing experiments under conditions wherein proteins may be immobilized insufficiently or wherein nucleic acids may bind insufficiently. Thus, it has not always been possible to obtain expected results by the conventional methods, because, for example, nucleic acids which bind by non-specific adsorption to beads, membranes or the like are also screened in addition to nucleic acids binding via the nucleic acid recognition sites of proteins, or sequence is determined under conditions wherein the percentage of binding nucleic acid molecules is insufficient due to insufficiency of selection cycles.

### SUMMARY OF THE INVENTION

It is an object of the present invention to solve the above problems of the prior art. The present invention provides a means of selecting nucleic acids, wherein the immobilization of proteins and the binding state of nucleic acids can be observed (or detected) in real-time, and thereby selection of nucleic acids can be carried out in a situation that the immobilization of polypeptides and the binding of the immobilized polypeptides to nucleic acids are assumed to be complete, and in particular, the protein and nucleic acid are substantially assumed to be bound to each other only via the nucleic acid recognition site of the polypeptide. This means makes it possible to determine a nucleotide sequence recognized by a polypeptide, or to carry out rapid and precise functional analysis of proteins or nucleic acids.

As a result of focused research, we found that the above problems could be solved by preparing a sensor chip modified by introducing NTA group onto a surface plasmon resonance sensor chip; immobilizing a polypeptide containing an oligo His tag via the NTA group; detecting and confirming the binding state of an immobilized polypeptide to a nucleic acid by a surface plasmon resonance method using the modified sensor chip; and then selecting a nucleic acid, and thereby we completed the present invention.

That is, the present invention relates to the following (1) to (6):

(1) A sensor chip for surface plasmon resonance measurement used for selecting a nucleic acid that binds to a polypeptide, wherein the chip has NTA group on its surface for immobilizing a polypeptide.

(2) A sensor chip for surface plasmon resonance measurement used for selecting a nucleic acid that binds to a polypeptide, wherein a polypeptide containing a His tag is immobilized via NTA group.

(3) The sensor chip of (1) or (2), wherein NTA group is introduced onto a surface plasmon resonance sensor chip having a reduced density of carboxymethyl group existing on its surface.

(4) The sensor chip of (1) or (2), wherein NTA group is introduced onto a surface plasmon resonance sensor chip having carboxymethyl group in such an amount capable of immobilizing a 400 to 600 RU of a protein of SEQ ID NO: 1 in an equilibrated state in measurement by a surface plasmon resonance method.

(5) A method for selecting a nucleic acid bound to a polypeptide by contacting a solution containing nucleic acids with a sensor chip, having NTA group on its surface and immobilizing His tags-containing polypeptides thereto, used for surface plasmon resonance measurement, which comprises selecting nucleic acids while detecting nucleic acid binding states to the immobilized polypeptides by a surface plasmon resonance method.

The present invention is explained in detail below.

The surface plasmon resonance method detects a slight change of refractive index of a thin film by light, which is caused by molecular reaction on a sensor chip made of a metal thin film such as gold. Real-time detection of increases and decreases in mass caused by addition and elimination of molecules is possible by this method.

In the present invention, a sensor chip having a thin film layer of carboxymethyldextran on a part of the surface of the metal thin film is used. The size of the chip itself is 8.9 cm×2.5 cm, and the size of the thin film layer portion of carboxymethyldextran is 0.7 cm×0.7 cm.

In the present invention, for immobilizing polypeptides onto a sensor chip, NTA group are introduced onto the sensor chip, and then polypeptides are immobilized via the NTA group. Specifically, the NTA group is introduced onto a sensor chip by an amine-coupling reaction which comprises activating the carboxymethyl group of the above carboxymethyldextran using N-hydroxy succinimide (NHS), N-ethyl-N′-(3-dimethylaminopropyl) carbodiimide hydrochloride (EDC) etc., and reacting with ethanolamine and N-(5-amino-1-carboxypentyl)-iminodiacetic acid to react, and then blocking with ethanolamine. In other words, the NTA group in the present invention is the group introduced by amine coupling of N-(5-amino-1-carboxypentyl)-iminodiacetic acid, and has the following partial structural formula:

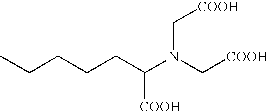

To immobilize polypeptides using NTA group, metal ions such as Ni²⁺ are coordinately bound to NTA group, while oligo His tags are added to proteins to be immobilized, and then the added His tags are coordinately bound to Ni²⁺-NTA group to immobilize the polypeptides.

Various kinds of sensor chips having different densities of surface carboxymethyl group have been known so far. A preferred sensor chip used in the present invention has a small amount of the surface carboxymethyl group, relatively. It is preferable that a sensor chip having a reduced density of carboxymethyl group existing on the surface is used to introduce NTA group onto the sensor chip and immobilize the polypeptides.

The reason for this is as follows. Not all the carboxymethyl group on the thin film layer of carboxymethyldextran are not reacted when NTA group are introduced, and as a result the carboxymethyl group remain on the chip on which polypeptides are immobilized. When many carboxymethyl group that have a negative charge exist on the surface, the negative charge of the sensor chip surface increases and it results in inhibiting binding with nucleic acids that have similarly a negative charge by their electric repulsion. Therefore, advantageous results can be obtained in the present invention by using a sensor chip having a low density of carboxymethyl group, introducing NTA group, and immobilizing polypeptides thereto so as to reduce the amount of remaining carboxymethyl group.

To select a nucleic acid that binds to a polypeptide in the present invention, for example, a sensor chip separately prepared to have polypeptides immobilized thereto is set in a surface plasmon resonance measurement system, and a sample solution containing nucleic acids is allowed to flow over the chip, and unbound nucleic acids are washed off while detecting the binding state simultaneously, and then only nucleic acids bound to polypeptides are dissociated and collected. At this time, it is more advantageous to select nucleic acids while detecting not only the binding state of nucleic acids to the immobilized polypeptides, but also the progress of immobilization of polypeptides using a surface plasmon resonance measurement system. For example, after a sensor chip is set in a surface plasmon resonance measurement system, the polypeptides-immobilization step and the nucleic acids-binding step are performed sequentially with the progress of these two steps detected (or observed).

According to the present invention, the state of progress of the binding of nucleic acids to polypeptides and of other steps can be detected in real-time using a surface plasmon resonance measurement system. Thus, a risk of proceeding with experiments with insufficient immobilization of polypeptides or insufficient binding of nucleic acids to polypeptides can be avoided. Moreover, for example, the experimental conditions can be further adjusted when these reactions are insufficient. Thus, it becomes possible to select substantially only nucleic acids which bind by recognizing the nucleic acid recognition site of protein.

Specific examples of the polypeptide to be immobilized in the present invention include transcription factors, replication factors and recombination factors, and specific examples of nucleic acids to be selected by their binding to these factors include DNA and RNA. For example, in the present invention, a solution of the nucleic acids molecules including various DNAs is allowed to flow over a sensor chip, nucleic acids that do not bind to polypeptides are washed off, and then bound nucleic acids are eluted together with the polypeptides. The eluted nucleic acids are amplified by PCR, and then allowed to flow again over the sensor chip having proteins immobilized thereto. Subsequently, bound nucleic acids are eluted in the same manner as described above, and then amplified by PCR. After repetition of this cycle several times, nucleic acids bound to polypeptides are selected and confirmed as binding to polypeptides, and then collected and purified. Then, the nucleic acid is subjected to determination of the sequence or the like.

In the present invention, more advantageous results can be obtained when a sensor chip having a reduced density of carboxymethyl group existing on the surface is used for introduction of NTA group. A commercially available surface plasmon resonance measurement sensor chip is denoted to have a reduced level of carboxymethyl group, however, no detailed description is given for the density or the amount. Hence, an experiment is conducted for verification as follows.

(Experiment for Verifying Surface Carboxymethyl Group)

A B1 sensor chip (Biacore; the size of carboxymethyldextran portion is 0.7 cm×0.7 cm) denoted to have a reduced level of carboxymethyl group and a normal CM5 chip (Biacore; the size of carboxymethyldextran portion is 0.7 cm×0.7 cm) were respectively installed in a surface plasmon resonance measurement system BIACORE X (Biacore). Then, extra pure water was kept flowing at a rate of 5 μl/min on the chips. The internal temperature was set at 25° C. N-hydroxysuccinimide(NHS) and N-ethyl-N′-(3-dimethylaminopropyl)carbodiimide hydrochloride (EDC) in an amine coupling kit (Biacore) were dissolved at a concentration of 100 mM and 400 mM, respectively, and then equivalent volumes thereof were mixed. After the mixed solution was allowed to flow for 7 minutes over the chip, and a solution (50 mM boric acid (pH 8.5), 150 mM NaCl) containing the DNA binding domain (SEQ ID NO: 1) of plant protein NtERF2 dissolved therein at a concentration of 10 μM was allowed to flow for 7 minutes, and then ethanolamine solution in the amine coupling kit (Biacore) was allowed to flow for a further 7 minutes. Because proteins were immobilized via carboxymethyl group, the determined amount of immobilized proteins reflects the amount of carboxymethyl group existing in the carboxymethyldextran portion, or reflects the density (i.e., the amount of the groups per unit area). The result is shown in FIG. 1. As shown in FIG. 1, immobilization of protein onto normal CM5 chip caused an increased mass corresponding to 3139 RU. In contrast, in case of B1 chip, mass increase observed was 502 RU (relative amount 17%) at most (after immobilization to B1 chip was equilibrated).

In the present invention, using B1 chip is actually more advantageous to bind with nucleic acids, compared to using CM5 chip. On the other hand, since introduction of NTA group is performed via carboxymethyl group in the present invention, a certain amount or more of carboxyl groups are required. Therefore, it can be concluded by collective examination of these facts that a preferable result can be obtained when surface carboxymethyl group on the sensor chip to be used for introducing NTA group according to the present invention exist in such an amount which allows immobilization of protein of SEQ ID NO: 1 corresponding to 400 to 600 RU in the equilibrium state as measured by a surface plasmon resonance method.

This specification includes part or all of the contents as disclosed in the specification of Japanese Patent Application No. 2002-149330, which is a priority document of the present application.

### BRIEF DESCRIPTION OF THE DRAWINGS

FIG. 1 shows the immobilization-state of the polypeptide represented by SEQ ID NO: 1 to B1 chip (solid line) and to CM5 chip (dotted line) as measured by surface plasmon resonance real-time measurement.

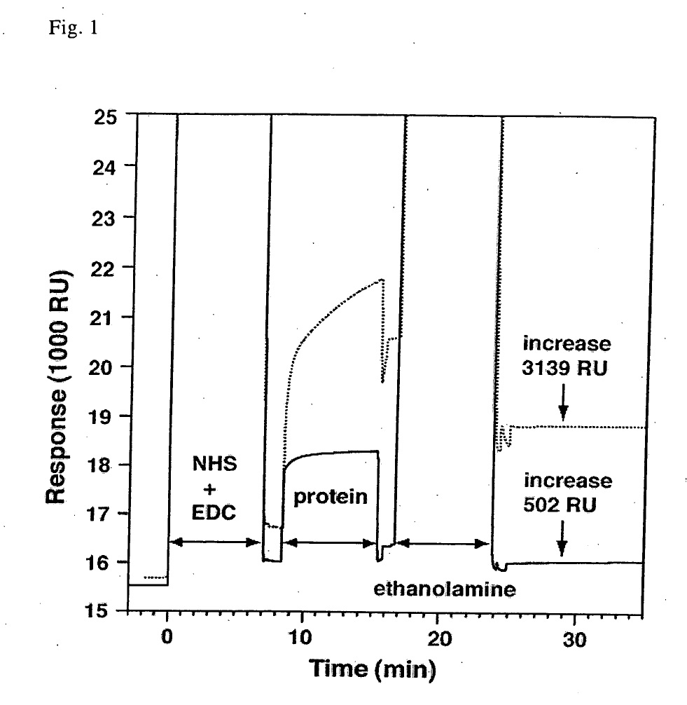

FIG. 2 shows the immobilization-states of protein to a sensor chip (a) and binding-states of nucleic acids (b) as measured by surface plasmon resonance real-time measurement.

### DESCRIPTION OF THE PREFERRED EMBODIMENTS

(Modification of Sensor Chip)

B1 chip (Biacore), a type of surface plasmon resonance sensor chip having a reduced density of carboxymethyl group that cause a negative charge, was installed to a surface plasmon resonance measurement system BIACORE X (Biacore), and then extra pure water was kept flowing at a rate of 5 ml/min on the chip.

The internal temperature was set at 25° C. N-hydroxysuccinimide and N-ethyl-N′-(3-dimethylaminopropyl)carbodiimide hydrochloride in an amine coupling kit (Biacore) were dissolved at a concentration of 100 mM and 400 mM, respectively, and then equivalent volumes thereof were mixed. The mixed solution was allowed to flow over the chip for 7 minutes. Further, a solution containing 50 mM N-(5-amino-1-carboxypentyl)-iminodiacetic acid (DOJINDO), 50 mM boric acid (pH 8.5) and 150 mM NaCl was allowed to flow for 7 minutes, and then an ethanolamine solution in the amine coupling kit (Biacore) was allowed to flow for 7 minutes over the chip. Thus, a modified sensor chip having NTA group on its surface and a reduced density of carboxymethyl group was prepared. The prepared sensor chip was removed from the system, and then stored in a refrigerator.

(System for Selecting Nucleic Acid Sequence)

The above sensor chip was installed into a surface plasmon resonance measurement system BIACORE X (Biacore), so that the system for selecting a nucleic acid sequence of the present invention was composed.

(Immobilization of Protein)

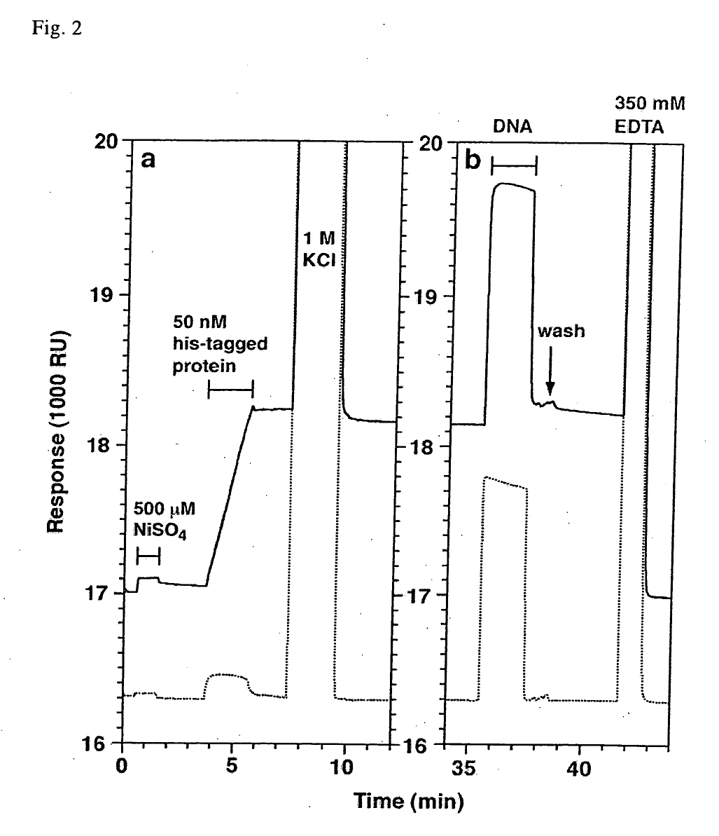

The internal temperature of the system was set at 25° C. Then, buffer A (25 mM (4-(2-hydroxyethyl)-1-piperazinyl) ethanesulphonic acid (pH7.0), 40 mM KCl, 0.2 mM ethylenediamine tetraacetic acid (EDTA), 0.005% Tween20) was kept flowing at a rate of 10 μl/min. NiSO₄ was dissolved at a concentration of 500 μM in buffer A, and then the solution was allowed to flow over the chip for 1 minute, thereby allowing Ni²⁺ ions to bind to NTA group. Polypeptides containing oligo His tags (for example: DNA binding domain of plant protein NtERF2=SEQ ID NO: 1) were dissolved at a concentration of 50 nM in buffer A, and then the solution was allowed to flow over the chip for 2 minutes, thereby allowing the oligo His tags in the polypeptides to coordinately bind to Ni²⁺-NTA group. Next, KCl was dissolved at a concentration of 1 M in buffer A, the solution was allowed to flow over the chip for 1 minute, and then polypeptides not bound coordinately but weakly bound electrostatically were washed off, thereby completing immobilization of polypeptides. During this procedure, the binding of each molecule onto the chip was quantitatively monitored by surface plasmon resonance real-time measurement. This observation is shown in FIG. 2(a). In addition, the sensor chip used in the present invention has two separated sections, and measurement simultaneously using the two sections is possible. Solid lines in the figure represent resonance responses in the NTA group-introduced section, and dotted lines represent resonance responses for the section with no NTA group introduced.

(Preparation of Solution of Molecular Association of Nucleic Acids Having Randomized Sequences)

A single stranded DNA (SEQ ID NO: 2: “n” represents a randomized portion, that is, any one of 4 types of nucleotides) having a sequence partially randomized using a nucleotide mixture upon chemical synthesis and 3′ primer (SEQ ID NO: 3) were mixed, and then the mixture was subjected to an elongation reaction using DNA polymerase I (Boerhinger Mannheim), thereby preparing a double-stranded DNA having the single stranded DNA of SEQ ID NO: 2 as one half. The prepared double stranded DNA was purified using QIAquick Nucleotide Removal Kit (QIAGEN), and then dissolved in buffer A.

(Binding of Nucleic Acids to and Dissociation of Nucleic Acids from Polypeptide-Immobilized Sensor Chip)

The internal temperature of the system to which polypeptides had been immobilized was set at 25° C. Then, buffer A was kept flowing at a rate of 10 μl/min. A solution of the double stranded DNAs moleculars having randomized sequences prepared by polymerase elongation was allowed to flow over the chip for 2 minutes, and then nucleic acid molecules that had not been bound to proteins were washed off from the chip surface. EDTA was dissolved at a concentration of 350 mM into buffer A, and then the solution was allowed to flow over the chip for 1 minute. Thereby, as Ni²⁺ ions bound to the NTA group of the chip dissociated, coordinately bound proteins and nucleic acid molecules bound to the proteins were also eluted. During this procedure, the binding of each molecule onto the chip was quantitatively monitored by surface plasmon resonance real-time measurement.

This observation is shown in FIG. 2(b). In addition, the sensor chip used in the present invention has two separated sections, and measurement simultaneously using the two sections is possible. Solid lines in the figure represent resonance responses in the NTA group-introduced section, and dotted lines represent resonance responses for the section with no NTA group introduced.

(Amplification of DNA and Selection Cycle)

7 mM MgCl₂, and primer DNAs (SEQ ID NO: 3 and 4) were added to the above eluted nucleic acid molecules, and this was subjected to PCR reaction on a thermal cycler (BioRad, ICycler) using pyroBest DNA polymerase (TAKARA SHUZO CO., LTD.). PCR reaction condition was 15 cycles consisted of 95° C. for 1 minute, 55° C. for 0.5 minute, and 72° C. for 0.5 minute. The solution of amplified nucleic acids was purified using QIAquick Nucleotide Removal Kit (QIAGEN), and then the purified product was dissolved in buffer A. The solution was again applied to the above polypeptide-immobilized sensor chip, and then binding/dissociation step (selection step) were carried out in a similar manner. This cycle (a set of amplification and selection step) was repeated for 7 times.

(Final Purification and Sequencing of Nucleic Acid Molecules)

Final purification was carried out as follows. The solution of nucleic acids collected after 7 cycles of the above selection and amplification was subjected to 8% polyacrylamide gel electrophoresis, and then bands were excised with a cutter knife. The purified product was cloned into pUC 119 plasmid using a restriction enzyme Sma I (TAKARA SHUZO). Then the plasmid was transformed into Escherichia coli strain DH5α (TAKARA SHUZO), and 40 to 50 colonies were picked up and then cultured in LB media. The plasmid DNA produced within bacteria was purified using a purification kit (Centricep (Princeton Separation)). The purified product was subjected to sequencing using a DNA sequencer ABI310 Genetic Analyzer (Perkin-Elmer). The result is shown in Table 1 below.

TABLE 1

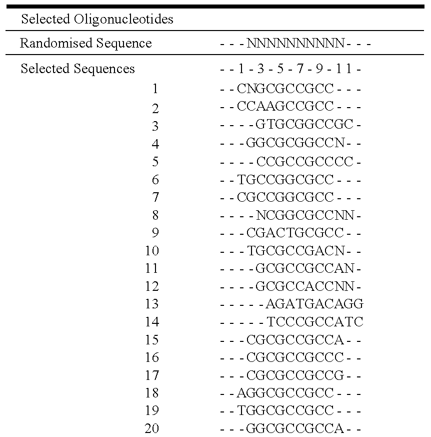

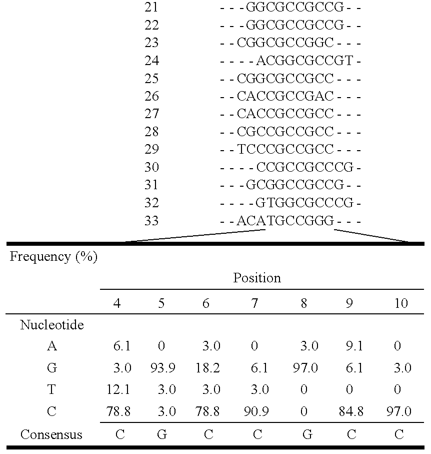

It is clear that GCCGCC sequence, the recognition sequence of the transcription factor NtERF2, was selected as shown in the result, and accordingly the effectiveness of this system was demonstrated.

All publications, patents and patent applications cited herein are incorporated herein by reference in their entirety.

### INDUSTRIAL APPLICABILITY

As is clear from the above description, according to the present invention, nucleic acids that recognize and bind to the nucleic acid recognition sites of polypeptides can be selected rapidly with extremely high accuracy by fast and simple procedures. Hence, the present invention can greatly contribute to elucidating the nucleotide sequence of a nucleic acid to be recognized at the recognition site or elucidating the functions of polypeptides and nucleic acids.

What is claimed is:

1. A sensor chip for surface plasmon resonance measurement used for selecting a nucleic acid that binds to a polypeptide, wherein the chip has NTA group on its surface for immobilizing a polypeptide.

2. A sensor chip for surface plasmon resonance measurement used for selecting a nucleic acid that binds to a polypeptide, wherein a polypeptide containing a His tag is immobilized via NTA group.

3. The sensor chip of claim 1 or 2, wherein NTA group is introduced onto a surface plasmon resonance sensor chip having a reduced density of carboxymethyl group existing on its surface.

4. The sensor chip of claim 1 or 2, wherein NTA group is introduced onto a surface plasmon resonance sensor chip having carboxymethyl group in such an amount capable of immobilizing a 400 to 600 RU of a protein of SEQ ID NO: 1 in an equilibrated state in measurement by a surface plasmon resonance method.

5. A method for selecting a nucleic acid bound to a polypeptide by contacting a solution containing nucleic acids with a sensor chip, having NTA group on its surface and immobilizing His tags-containing polypeptides thereto, used for surface plasmon resonance measurement, which comprises selecting nucleic acids while detecting nucleic acid binding states to the immobilized polypeptides by a surface plasmon resonance method.


SEQUENCE LISTING (37 sequences)

SEQ ID NO: 1 - protein, 100 aa, Arabidopsis thaliana
MGHHHHHHHHHHSSGHIEGRHMTAQAVVPKGRHYRGVRQRPWGKFAAGIRDPAKNGARVWLGTYETAEEAALAAYDKAAYRMRGSKALLNFPHRIGLNEP

SEQ ID NO: 2 - DNA, 60 bp, Artificial Sequence
  Description of Artificial Sequence Synthetic oligonucleotide
ctgtcagtgatgcatatgaacgaatnnnnnnnnnnaatcaacgacattaggatccttagc

SEQ ID NO: 3 - DNA, 20 bp, Artificial Sequence
  Description of Artificial Sequence PCR primer
gctaaggatcctaatgtcgt

SEQ ID NO: 4 - DNA, 20 bp, Artificial Sequence
  Description of Artificial Sequence PCR primer
ctgtcagtgatgcatatgaa

SEQ ID NO: 5 - DNA, 10 bp, Artificial Sequence
  Description of Artificial Sequence Synthetic oligonucleotide
cngcgccgcc

SEQ ID NO: 6 - DNA, 10 bp, Artificial Sequence
  Description of Artificial Sequence Synthetic oligonucleotide
ccaagccgcc

SEQ ID NO: 7 - DNA, 10 bp, Artificial Sequence
  Description of Artificial Sequence Synthetic oligonucleotide
gtgcggccgc

SEQ ID NO: 8 - DNA, 10 bp, Artificial Sequence
  Description of Artificial Sequence Synthetic oligonucleotide
ggcgcggccn

SEQ ID NO: 9 - DNA, 10 bp, Artificial Sequence
  Description of Artificial Sequence Synthetic oligonucleotide
ccgccgcccc

SEQ ID NO: 10 - DNA, 10 bp, Artificial Sequence
  Description of Artificial Sequence Synthetic oligonucleotide
tgccggcgcc

SEQ ID NO: 11 - DNA, 10 bp, Artificial Sequence
  Description of Artificial Sequence Synthetic oligonucleotide
cgccggcgcc

SEQ ID NO: 12 - DNA, 10 bp, Artificial Sequence
  Description of Artificial Sequence Synthetic oligonucleotide
ncggcgccnn

SEQ ID NO: 13 - DNA, 10 bp, Artificial Sequence
  Description of Artificial Sequence Synthetic oligonucleotide
cgactgcgcc

SEQ ID NO: 14 - DNA, 10 bp, Artificial Sequence
  Description of Artificial Sequence Synthetic oligonucleotide
tgcgccgacn

SEQ ID NO: 15 - DNA, 10 bp, Artificial Sequence
  Description of Artificial Sequence Synthetic oligonucleotide
gcgccgccan

SEQ ID NO: 16 - DNA, 10 bp, Artificial Sequence
  Description of Artificial Sequence Synthetic oligonucleotide
gcgccaccnn

SEQ ID NO: 17 - DNA, 10 bp, Artificial Sequence
  Description of Artificial Sequence Synthetic oligonucleotide
agatgacagg

SEQ ID NO: 18 - DNA, 10 bp, Artificial Sequence
  Description of Artificial Sequence Synthetic oligonucleotide
tcccgccatc

SEQ ID NO: 19 - DNA, 10 bp, Artificial Sequence
  Description of Artificial Sequence Synthetic oligonucleotide
cgcgccgcca

SEQ ID NO: 20 - DNA, 10 bp, Artificial Sequence
  Description of Artificial Sequence Synthetic oligonucleotide
cgcgccgccc

SEQ ID NO: 21 - DNA, 10 bp, Artificial Sequence
  Description of Artificial Sequence Synthetic oligonucleotide
cgcgccgccg

SEQ ID NO: 22 - DNA, 10 bp, Artificial Sequence
  Description of Artificial Sequence Synthetic oligonucleotide
aggcgccgcc

SEQ ID NO: 23 - DNA, 10 bp, Artificial Sequence
  Description of Artificial Sequence Synthetic oligonucleotide
tggcgccgcc

SEQ ID NO: 24 - DNA, 10 bp, Artificial Sequence
  Description of Artificial Sequence Synthetic oligonucleotide
ggcgccgcca

SEQ ID NO: 25 - DNA, 10 bp, Artificial Sequence
  Description of Artificial Sequence Synthetic oligonucleotide
ggcgccgccg

SEQ ID NO: 26 - DNA, 10 bp, Artificial Sequence
  Description of Artificial Sequence Synthetic oligonucleotide
ggcgccgccg

SEQ ID NO: 27 - DNA, 10 bp, Artificial Sequence
  Description of Artificial Sequence Synthetic oligonucleotide
cggcgccggc

SEQ ID NO: 28 - DNA, 10 bp, Artificial Sequence
  Description of Artificial Sequence Synthetic oligonucleotide
acggcgccgt

SEQ ID NO: 29 - DNA, 10 bp, Artificial Sequence
  Description of Artificial Sequence Synthetic oligonucleotide
cggcgccgcc

SEQ ID NO: 30 - DNA, 10 bp, Artificial Sequence
  Description of Artificial Sequence Synthetic oligonucleotide
caccgccgac

SEQ ID NO: 31 - DNA, 10 bp, Artificial Sequence
  Description of Artificial Sequence Synthetic oligonucleotide
caccgccgcc

SEQ ID NO: 32 - DNA, 10 bp, Artificial Sequence
  Description of Artificial Sequence Synthetic oligonucleotide
cgccgccgcc

SEQ ID NO: 33 - DNA, 10 bp, Artificial Sequence
  Description of Artificial Sequence Synthetic oligonucleotide
tcccgccgcc

SEQ ID NO: 34 - DNA, 10 bp, Artificial Sequence
  Description of Artificial Sequence Synthetic oligonucleotide
ccgccgcccg

SEQ ID NO: 35 - DNA, 10 bp, Artificial Sequence
  Description of Artificial Sequence Synthetic oligonucleotide
gcggccgccg

SEQ ID NO: 36 - DNA, 10 bp, Artificial Sequence
  Description of Artificial Sequence Synthetic oligonucleotide
gtggcgcccg

SEQ ID NO: 37 - DNA, 10 bp, Artificial Sequence
  Description of Artificial Sequence Synthetic oligonucleotide
acatgccggg